(19) United States
(12) Patent Application Publication
(10) Pub. No.: US 2015/0328348 A1
(43) Pub. Date: Nov. 19, 2015

(54) DRYING AND DISINFECTING SYSTEM, METHOD AND DEVICE FOR BABY BOTTLES AND OTHER ITEMS USING INFRARED LIGHT AND ULTRAVIOLET RADIATION

(71) Applicant: Jose Benjamin Colayco, Irvine, CA (US)

(72) Inventor: Jose Benjamin Colayco, Irvine, CA (US)

(21) Appl. No.: 14/276,330

(22) Filed: May 13, 2014

(51) Int. Cl. A61L 2/10; F26B 3/30; A61L 2/24

(52) U.S. Cl. CPC A61L 2/10; A61L 2/24; F26B 3/30

(57) ABSTRACT

A drying and disinfecting device utilizing ultraviolet radiation (UV) and infrared light (IR) to dry and disinfect baby bottles and other objects placed therein, the device comprising: a drying and disinfecting chamber for containing small objects to be dried and disinfected therein; an ultraviolet light source and an infrared light source operable to expose baby bottles and other objects to ultraviolet radiation; a protective UV-blocking viewing window; a convenience handle for portability; a user interface; a controller, the controller operable to: initiate a drying and disinfecting cycle in which the ultraviolet light source is caused to emit ultraviolet light in response to user input via the user interface; and cause the user interface to indicate a status of the device. The device creates significant convenience over and avoids the disadvantages of the traditional use of high heat, moisture and steam to dry and disinfect baby bottles and other objects.

### BACKGROUND OF THE INVENTION

The subject matter described herein relates generally to a system, device, and method of drying and disinfecting baby bottles and other objects using infrared light (IR) and ultraviolet radiation (UV).

Existing and traditional disinfecting methods for baby bottles depend on high temperatures, moisture and/or steam. The use of UV and IR light eliminates this dependency, increasing safety and convenience since UV and IR wavelengths do not require high temperatures that may cause burns, or moisture or steam that may create lime scale build-up. Traditional methods may also limit the types of objects that can be disinfected due to the exposure to high temperatures, moisture and/or steam. Objects that cannot be exposed to high heat, moisture and/or steam such as electronics (e.g. mobile phones), and other items (e.g. keys, wooden blocks, certain toys) can all be disinfected with UV light. The human immune system is a biological system including numerous processes and structures which combine to protect against disease. Properly functioning human immune systems are able to detect and fight viruses, parasites, bacteria, and other harmful pathogens in order to keep the human body healthy. Compromised or less than fully functional immune system function may lead to increased risk of illness and disease. Immune systems in infants may not be fully developed; therefore infants may be more susceptible to illness from pathogens than adults with fully functioning immune systems.

Ultraviolet (UV) radiation is a form of electromagnetic radiation with wavelengths shorter than visible light. Ultraviolet germicidal irradiation is a method of disinfection using ultraviolet light to kill microorganisms. Although multiple forms of ultraviolet radiation exist, short-wavelength ultraviolet radiation such as UV-C radiation is known to have germicidal properties and to effectively kill microorganisms.

Infrared Light (IR) is also a form of electromagnetic radiation with wavelengths shorter than visible light and is known to have drying and curing effects without damaging material. As such, it may be used to dry baby bottles and other objects while generating a relatively low amount of heat.

U.S. Pat. No. 7,165,562 teaches a washing device for an infant bottle (and associated parts, e.g. nipple and nipple cap). The device washes, dries and sterilizes (using UV light) the bottle after washing.

U.S. Pat. No. 7,165,562 teaches a portable device for sterilizing small objects using UV light.

While such UV-based sterilization devices are known, the enablements provided herein teach features and advantages heretofore untaught by the prior art, as will be clear to one of ordinary skill in the art

### SUMMARY OF THE INVENTION

Provided herein are embodiments of a device, system, and method of drying and disinfecting baby bottles and other objects using infrared light (IR) and ultraviolet light (UV).

Other features and advantages of the present invention will become apparent from the following more detailed description, taken in conjunction with the accompanying drawings, which illustrate, by way of example, the principles of the present invention.

### BRIEF DESCRIPTION OF THE SEVERAL VIEWS OF THE DRAWING(S)

Illustrated in the accompanying drawing(s) is at least one of the best mode embodiments of the present invention. In such drawing(s):

FIG. 1A illustrates an exemplary drying and disinfecting device with a door in a closed configuration in accordance with at least one embodiment of the present invention.

FIG. 1B illustrates an exemplary drying and disinfecting device with a door in an open configuration for a view of an interior chamber in accordance with at least one embodiment of the present invention.

FIG. 2 illustrates an exemplary display featuring status indicator in accordance with at least one embodiment of the present invention.

FIG. 3 illustrates an exemplary embodiment of the device during operation of a drying function using infrared light (IR).

FIG. 4 illustrates an exemplary embodiment of the device during operation of a disinfecting function using ultraviolet light (UV).

FIG. 5 illustrates an exemplary display of a “convenience” handle for portability and travel, and an area within the casing that houses a universal power supply that allows the apparatus to operate according to different power standards.

FIG. 6 illustrates an exemplary embodiment of a system diagram of the device including various components.

### DETAILED DESCRIPTION OF THE PREFERRED EMBODIMENTS

The above described drawing figures illustrate the described invention and method of use in at least one of its preferred, best mode embodiment, which is further defined in detail in the following description. Those having ordinary skill in the art may be able to make alterations and modifications to what is described herein without departing from its spirit and scope. While this invention is susceptible of embodiment in many different forms, there is shown in the drawings and will herein be described in detail a preferred embodiment of the invention with the understanding that the present disclosure is to be considered as an exemplification of the principles of the invention and is not intended to limit the broad aspect of the invention to the embodiment illustrated. All features, elements, components, functions, and steps described with respect to any embodiment provided herein are intended to be freely combinable and substitutable with those from any other embodiment unless otherwise stated. Therefore, it should be understood that what is illustrated is set forth only for the purposes of example and should not be taken as a limitation on the scope of the present invention.

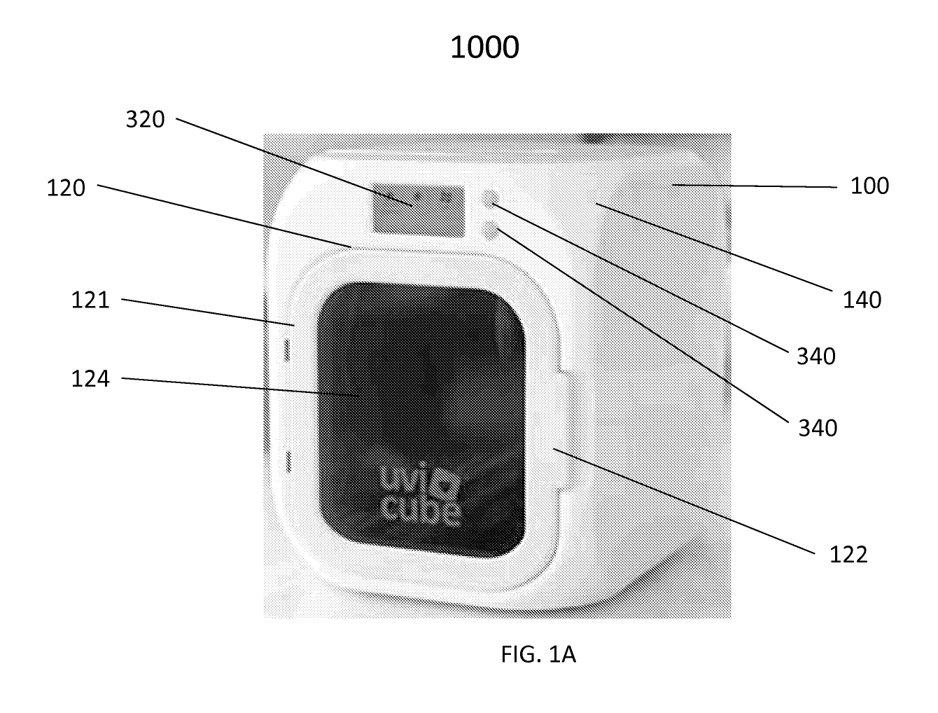

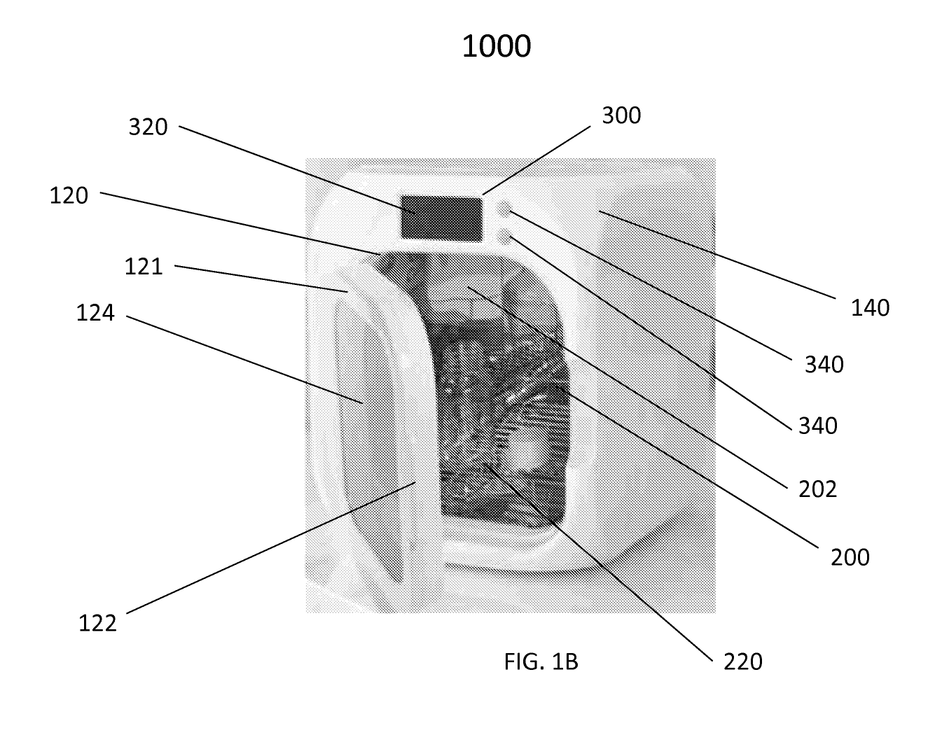

Turning to FIGS. 1A and 1B, an exemplary drying and disinfecting device is shown.

The drying and disinfecting device 1000 generally comprises a housing 100 made from UV radiation resistant material and having a plurality of walls, a top, a bottom, an interior and an exterior, the housing defining an internal chamber 200 accessible via an entry 120, the interior chamber 200 including one or more of an IR and UV light source (not shown).

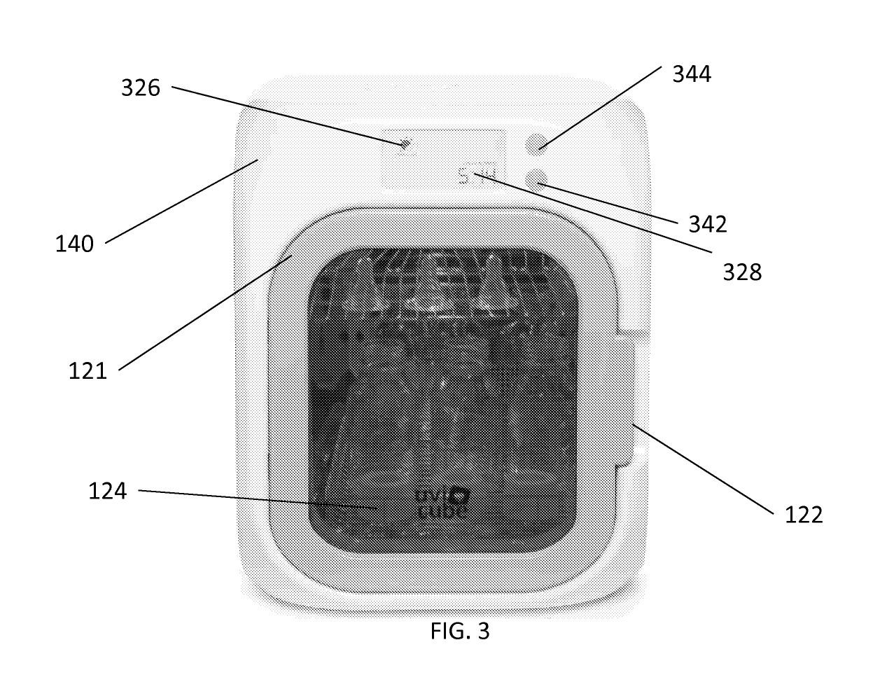

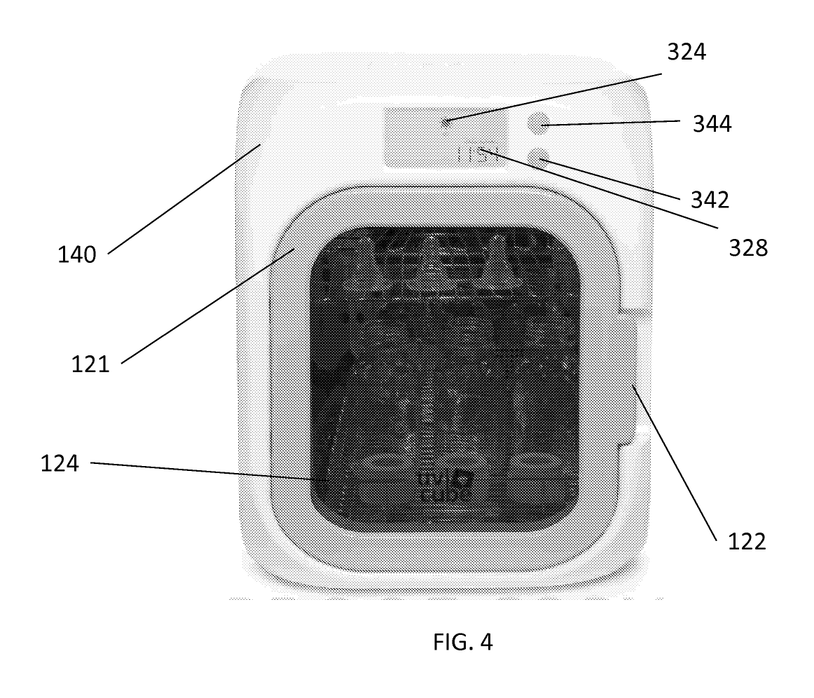

In operation, baby bottles and other objects are generally placed within the interior chamber 200 for timed exposure to the IR light source for drying baby bottles and other objects (as shown in FIG. 3), and timed exposure to the UV light source(as shown in FIG. 4), for disinfecting baby bottles and other objects.

The entry 120 provides selectable access to the internal chamber. In other words, the entry 120 is preferably configured such that the entry 120 may be opened (permitting access) or closed (prohibiting access). Accordingly, the entry 120 preferably includes a door 121 hingedly coupled to the housing 100. In some embodiments, the door 121 may slideably coupled to the housing and can be opened and closed via sliding relative thereto.

The entry 120 may further include a locking mechanism 122 for securing the entry 120 in a closed orientation. The locking mechanism 122 can include a latch that mechanically, magnetically, or electronically (or any combination thereof) secures the door 121 to the housing 100 when in the closed orientation. The locking mechanism 122 may include a safety feature that automatically pauses, interrupts or terminates any cycle when the door 121 is opened before the end of the cycle. A cycle may include a drying cycle, a disinfecting cycle or a combination cycle. The device 1000 may not begin operation or resume a paused, interrupted or terminated cycle unless the door 121 is properly closed and the locking mechanism 122 is engaged.

The device may further include one or more viewing window 124 for permitting the user visual access to the interior chamber 200. The one or more viewing window 124s may be integral to the housing, e.g. one or more of the walls, top, bottom, and/or may be integral to the entry 120. The viewing window 124 is constructed of UV radiation blocking, non-opaque material such as a substantially transparent plastic or glass that protects users from UV radiation but allows them to view an operation cycle and monitor the state of the contents within the device.

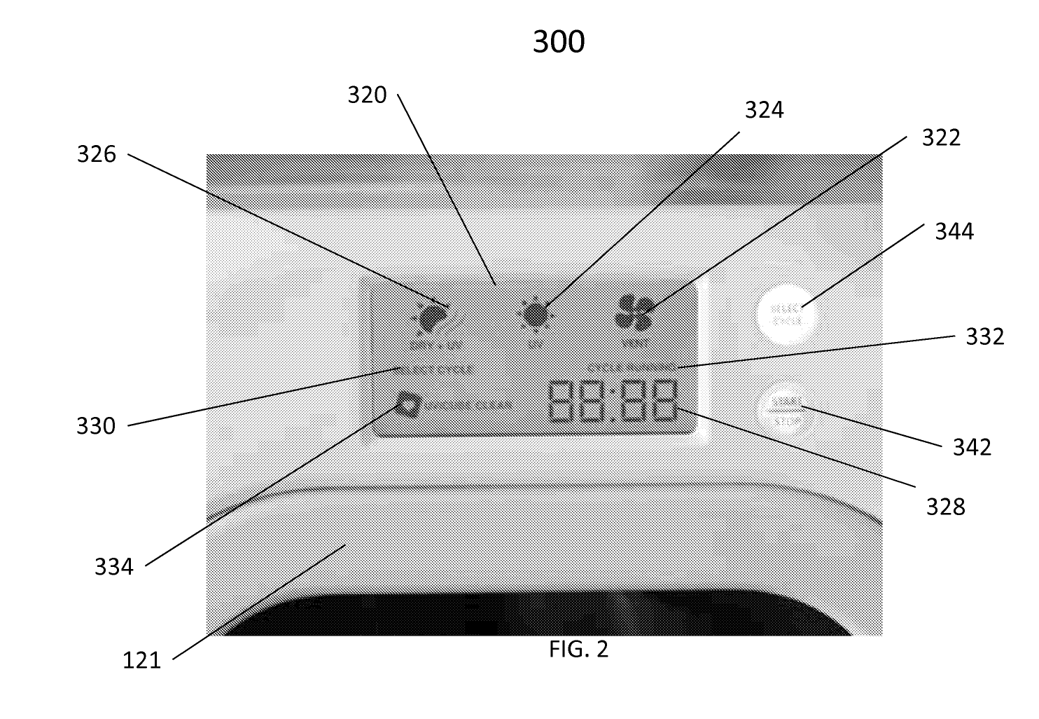

The device 1000 may further include a user interface 300 integral to the exterior of the housing 100 and described below in the description of FIG. 2

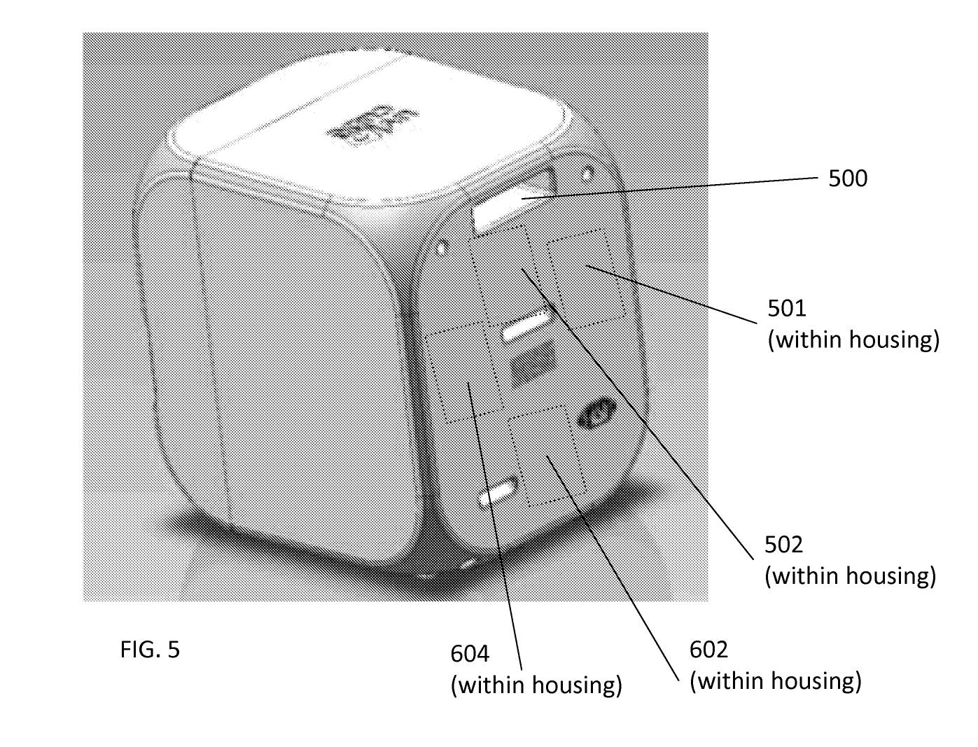

The device 1000 may further include one or more “convenience” handles 500, as shown in FIG. 5 for carrying the device. The handle 500 may provide users with a simple, convenient, and intuitive way to lift and transport the drying and disinfecting device. In at least one embodiment, the handle 500 may lay within a recess of the exterior of the device, promoting a sleek, uniformity to the device that discourages “snags.” In some embodiments, the handle 500 may be formed by the recess. In some embodiments, handle 500 may be transitioned from laying within the recess, to protruding from the recess so as to promote carrying. For example, the handle 500 may be hingedly coupled to the exterior of the housing so as to enable it to be “flipped” outwardly to protrude from the housing 100 and thereby promote carrying.

The housing 100 may further include chamfered corners 140 to enable simplified lifting and carrying of the device in accordance with the present invention. Chamfered corners 140 provide less of a chance of bumping against traditional corners as in a traditional “cube” or “box” configuration. In some embodiments, the housing 100 may be constructed of plastic, but other appropriate materials may be used.

As described herein, the interior chamber 200 includes one or more UV light or IR light sources for irradiating and drying the interior chamber and objects placed therein. Such UV light sources are known in the art. As shown in FIG. 1B, the interior chamber 200 may further include a highly reflective surface 202 for amplifying the effects of the irradiation and/or promoting irradiation of objects from all angles.

The interior chamber 200 may further include baby bottle or object support 220. The object support 220 may be a wire shelf, ledge or frame, suspended above a bottom surface of the interior chamber 200. Alternatively or additionally, it may hang from a top surface of the interior chamber 200. Moreover, the object support 220 may be removable from the interior chamber 200. Accordingly, in some embodiments, the interior chamber 200 includes side grooves for slideably accepting the object support 220 in similar fashion to how an oven rack is slideably mounted within an oven. The object support 220 may further comprise a plurality of legs or posts extending downwardly therefrom to suspend the object support 220 above the bottom surface of the interior chamber 200. In some embodiments, the object support 220 further includes a rack for securing baby bottles and objects in an upright or other orientation thereon.

The device may further include a fan 502 as shown in FIG. 5 to aid in the drying of baby bottles and other objects by the IR light source. The fan 502 and IR light source may operate independently of the UV light source, or may operate in combination therewith. Additionally, the fan 502 and IR light source may operate simultaneously or may operate at different intervals which may overlap during particular periods. In some embodiments, the fan includes a ‘silent’ mode or is substantially silent in operation.

The device further includes a controller 602 communicatively coupled to the IR light source 608 and UV-light source 606, controlled by buttons 344 and 342 located on the user interface 300, for controlling the operation thereof according to known methods. In some embodiments, operation of the device 1000 can be controlled through an application on a separate user device controlled wirelessly over the internet, wi-fi, blue-tooth or similar means. Examples of such user devices include mobile phones, laptops, desktops with wireless capabilities, tablets, wearable computers, and others.

The device further includes a universal power supply 501 as shown in FIG. 5. This universal power supply is able to accommodate voltages in the range of 110-220 volts (common worldwide power supply range). This provides simplicity for users travelling internationally to be able to use the device 1000 wherever they may be. The power supply is further electrically coupled to the controller, the UV and IR light sources, the fan 502 and the user interface 300 so as to power the operation thereof.

Turning to FIG. 2 an exemplary user interface 300 is shown and described. As described herein, the user interface 300 may comprise the display 320 and one or more buttons 344 and 342 permitting a user to control the operation of the device 1000. The device 1000 may further include a user interface 300 integral to the exterior of the housing 100.

The user interface 300 may comprise a display 320 and one or more buttons 340 permitting a user to control the operation of the device 1000. The display 320 preferably provides visual indications to the user regarding the current state of the device 1000 and/or other information. For example, the display 320 can indicate the time remaining in a disinfecting cycle, the current time of day, the status of an automated schedule, and options for changing the operational parameters of the device. In future embodiments, the display 320 can be a touch-screen display permitting the user to control the operation of the device 1000 therefrom. Further, control of the device 1000 can be accessed through an application controlled wirelessly over the internet, through Wi-Fi, blue-tooth or similar means through devices such as mobile phones, laptops, desktops with wireless capabilities, tablets, wearable computers, and others.

The display 320 provides visual indications to the user regarding the current state of the device and/or other information. Preferably, the display 320 is liquid crystal diode (LCD) display 320. In at least one embodiment, the display 320 is a touch-screen display 320 permitting the user to control the operation of the device therefrom. The display 320 may be a back-lit, liquid crystal diode (LCD) screen that allows for the interface 300 to be easily seen both in daylight and complete darkness.

As shown in FIG. 2, the display 320 may include device operational parameter indicators. In some embodiments, this includes indicators as to whether the device 1000 is operating in a vent mode, a UV mode or a dry+UV mode.

In some embodiments, when the device 1000 is in vent mode, a vent mode indicator 322 is illuminated in the display 320. Moreover, when the device 1000 is in vent mode, a dryer operates independently of the UV or IR light source, as discussed herein, to dry objects placed in device 1000. Vent mode may include operation of a dryer where the dryer is fan 520.

In some embodiments, when the device is in UV mode, a UV mode indicator 324 is illuminated in the display 320. Moreover, when the device is in UV mode, the UV light source may operate independently of the IR light source, as discussed herein, to irradiate the interior chamber and sanitize small objects placed therein.

In some embodiments, when the device is in dry+UV mode, a dry+UV mode indicator 326 is illuminated in the display 320. Moreover, when the device is in dry+UV mode, the fan 520 and the UV light source operate in conjunction (either simultaneously, or sequentially) to dry and sanitize small objects placed therein.

In some embodiments, the display 320 includes a time indicator 328. The time indicator may display the current time. In some embodiments, the time indicator may display the time remaining in a current operation, i.e. the drying, disinfecting and/or drying and disinfecting of the small objects placed within the device. Such operations may be referred herein as cycles in that the operations may occur for a predetermined period of time such that the small objects be dried and/or disinfected thereby.

In some embodiments, the display 320 includes a cycle select indicator 330. Cycle select indicator 330 may allow the user to independently select the UV, IR or vent cycles. The dry+UV mode may be used for a full cycle of wet baby bottles or other objects. The UV mode may be for a full cycle for dry baby bottles or other objects (or objects that cannot be exposed to moisture or high heat). The vent mode may be used to ventilate the chamber if necessary.

In some embodiments, the display 320 includes a cycle running indicator 332. The cycle running indicator is illuminated when a one of the aforementioned cycles is running In some embodiments, the display 320 includes a cycle complete indicator 334. The cycle complete indicator is illuminated when a cycle is complete.

As discussed herein, one or more buttons 344 and 342 permit the user to control the operation of the device as per the operational features described herein. A start/stop button 342 and a cycle select button 344 are preferably provided. In operation, the user activates the device by pressing the start/stop button 342—which, for example, begins the dry+UV cycle as a default. This allows a “one-button-press” convenience versus traditional drying and disinfecting methods. By pressing the cycle select button, the user can select which of the modes the device 1000 will operate under—e.g. dry, UV or dry+UV or vent. By pressing the start/stop button once the appropriate cycle is selected, the user can initiate the selected cycle. By pressing the start/stop button while a cycle is running, the user can pause, interrupt or terminate the cycle in progress.

In some embodiments, the entry 120 includes a sensor for sensing whether the entry 120 is open or closed. The sensor may be communicatively coupled to the controller so as to communicate to the controller whether the entry 120 is open or closed. In some embodiments, when the entry 120 is opened during a cycle, the sensor communicates that information to the controller, which in turn stops the cycle until resumed. In some embodiments, when a cycle is stopped due to the entry 120 being opened mid-cycle, the controller automatically resumes the cycle in progress. In some embodiments, the controller requires user input—e.g. pressing of the start/stop button—before resuming the cycle in progress.

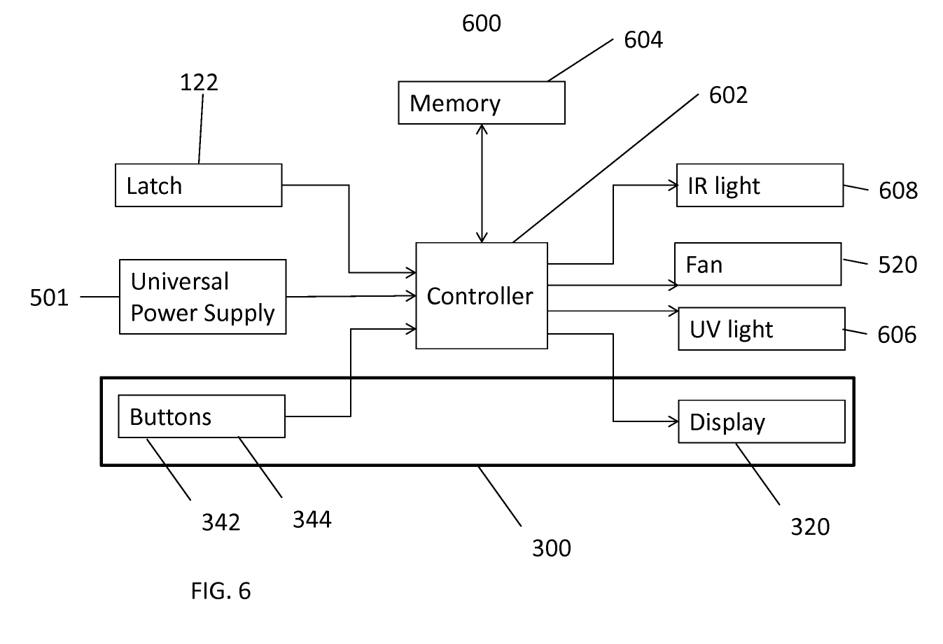

Turning to FIG. 6, an example embodiment of a system diagram is shown in accordance with the present invention. In the example embodiment controller 602 is shown along with its interaction with various components in device 1000. In the example embodiment controller 602 is in electrical communication with each of the other components. Controller 602 in an example embodiment is a hardware chip such as a central processing unit (CPU). Controller 602 carries out the instructions of a computer program using computer logic, arithmetic, input output operations and others.

Controller 602 may receive direct input from a user via the user interacting with buttons 324, 344 or opening or closing latch 122. In some embodiments, after receiving input such as a cycle select from a user, controller may access memory 604 in order to determine the length of a particular cycle or other parameters. Controller 602 may then use the parameters to activate IR light 608, UV light 606, Fan 520, or a combination of these components specific to the cycle selected. Controller 602 may cause display 320 to display the current status of the device such as a current cycle, the current time, or others. After completion of a cycle, controller 602 may update display 320 with a current status of the device and deactivate IR light 608, UV light 606, Fan 520 or the combination selected. In various embodiments additional hardware components may be added or omitted as desired.

The enablements described in detail above are considered novel over the prior art of record and are considered critical to the operation of at least one aspect of the invention and to the achievement of the above described objectives. The words used in this specification to describe the instant embodiments are to be understood not only in the sense of their commonly defined meanings, but to include by special definition in this specification: structure, material or acts beyond the scope of the commonly defined meanings Thus if an element can be understood in the context of this specification as including more than one meaning, then its use must be understood as being generic to all possible meanings supported by the specification and by the word or words describing the element.

The definitions of the words or drawing elements described herein are meant to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements described and its various embodiments or that a single element may be substituted for two or more elements in a claim.

Changes from the claimed subject matter as viewed by a person with ordinary skill in the art, now known or later devised, are expressly contemplated as being equivalents within the scope intended and its various embodiments. Therefore, obvious substitutions now or later known to one with ordinary skill in the art are defined to be within the scope of the defined elements. This disclosure is thus meant to be understood to include what is specifically illustrated and described above, what is conceptually equivalent, what can be obviously substituted, and also what incorporates the essential ideas.

The scope of this description is to be interpreted only in conjunction with the appended claims and it is made clear, here, that the named inventor believes that the claimed subject matter is what is intended to be patented.

What is claimed is:

1. A disinfecting device for objects utilizing Infrared light (IR) and ultraviolet radiation (UV), the device comprising:
a disinfecting chamber for containing objects to be dried and disinfected therein;
an ultraviolet light source operable to expose the objects to ultraviolet radiation;
a user interface indicating the status of the device;
a handle; and
a controller, the controller operable to:
initiate a disinfecting cycle in which the ultraviolet light source is caused to emit ultraviolet light in response to user input via the user interface.

2. The disinfecting device of claim 1, wherein the process includes drying and disinfecting.

3. The disinfecting device of claim 1, wherein objects include baby bottles.

4. The disinfecting device of claim 1, further comprising an infrared light source operable to expose objects to infrared wavelengths to promote drying.

5. The disinfecting device of claim 1, further comprising a viewing window allowing users to view the contents of the device while protecting the users from UV radiation.

6. The disinfecting device of claim 1, wherein indicating the status of the device includes indicating time and cycle data.

7. The disinfecting device of claim 1, wherein the controller is further operable to:
initiate a drying cycle in which the infrared light source is caused to emit infrared light in response to user input via the user interface.

8. The drying and disinfecting device of claim 1, wherein the device further includes a universal power supply for accepting 110V-240V and providing power to the device therefrom.

9. The drying and disinfecting device of claim 1, wherein the user interface includes a liquid crystal display that is viewable and operable in daylight and darkness.

10. The drying and disinfecting device of claim 1, wherein the device further includes means for locking the disinfecting chamber when in use.

11. The drying and disinfecting device of claim 1, further comprising an entry permitting access to the disinfecting chamber; wherein the controller is further operable to cause the ultraviolet light source to cease emitting ultraviolet light when the entry is opened during a cycle.

12. The drying and disinfecting device of claim 1, wherein the user interface displays a cycle timer showing a remaining time left in the disinfecting cycle.

13. The drying and disinfecting device of claim 1, further comprising one or more handles for carrying the device.

14. The drying and disinfecting device of claim 1, wherein a single button activation of the drying and the disinfecting cycle is a default cycle without selecting a cycle.
